(19) United States
(12) Patent Application Publication
(10) Pub. No.: US 2015/0376092 A1
(43) Pub. Date: Dec. 31, 2015

(54) RECOVERING H2 AND C2+ FROM FUEL GAS VIA USE OF A SINGLE-STAGE PSA AND SENDING PSA TAIL GAS TO GAS RECOVERY UNIT TO IMPROVE STEAM CRACKER FEED QUALITY

(71) Applicant: UOP LLC, Des Plaines, IL (US)

(72) Inventors: Robert E. Tsai, Arlington Heights, IL (US); Xin X. Zhu, Long Grove, IL (US); Ronald J. Long, Arlington Heights, IL (US)

(21) Appl. No.: 14/747,655

(22) Filed: Jun. 23, 2015

Related U.S. Application Data
(60) Provisional application No. 62/017,826, filed on Jun. 26, 2014.

(51) Int. Cl. C07C 7/11; C10L 3/10; B01D 53/047; C01B 3/02

(52) U.S. Cl. CPC C07C 7/11; C01B 3/02; C10L 3/101; B01D 53/047; C10L 2290/541; C10L 2290/545; B01D 2256/16; B01D 2256/245; B01D 2256/00

(57) ABSTRACT

The invention provides a process for treating a gas stream comprising hydrogen, methane and C₂+ hydrocarbons comprising sending the gas stream to a pressure swing adsorption unit to produce a first purified gas stream comprising more than 99 mol % hydrogen and a second purified gas stream comprising hydrogen, methane and C₂+ hydrocarbons, and sending the second purified gas stream to a gas plant to be separated into a plurality of streams.

### CROSS-REFERENCE TO RELATED APPLICATION

This application claims priority from Provisional Application No. 62/017,826 filed Jun. 26, 2014, the contents of which are hereby incorporated by reference.

### BACKGROUND OF THE INVENTION

The invention increases the amount of C₂+ hydrocarbons going to a steam cracker instead of fuel gas.

### SUMMARY OF THE INVENTION

The invention provides a process for treating a gas stream comprising hydrogen, methane and C₂+ hydrocarbons (such as ethane and larger hydrocarbons) comprising sending a gas stream to at least one pressure swing adsorption unit to produce a high quality hydrogen stream and a second gas stream comprising hydrogen, methane and C₂+ hydrocarbons and sending the second gas stream comprising hydrogen, methane and C₂+ hydrocarbons to a gas recovery unit (“gas plant”) to be separated into a plurality of product streams.

### BRIEF DESCRIPTION OF THE DRAWINGS

FIG. 1 shows a base gas processing flow scheme.

FIG. 2 shows a flow scheme with pressure swing adsorption tail gas sent to a gas plant.

FIG. 3 shows an alternative flow scheme with lean gas from a gas plant sent to a two-stage pressure swing adsorption train.

### DETAILED DESCRIPTION OF THE INVENTION

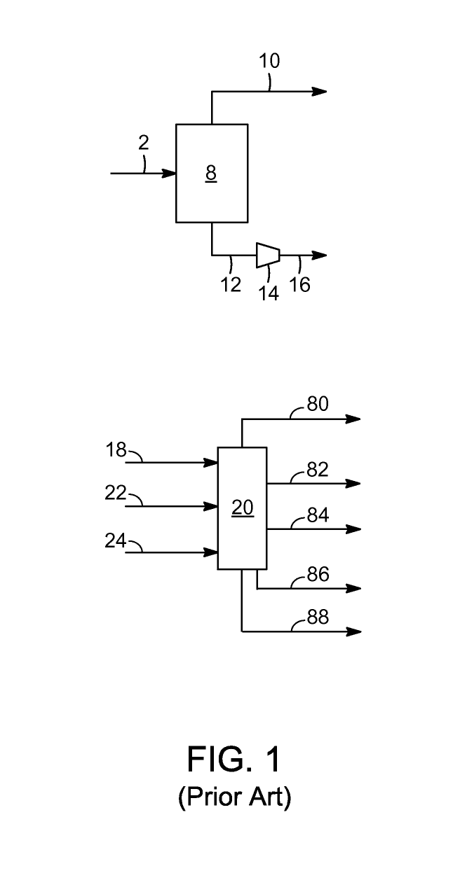

An example base scheme for a gas processing section within an integrated refinery and steam cracker complex is shown in FIG. 1. This section generates some high-purity hydrogen and fuel gas, but its main value is in the preparation of feed to the steam cracker. In steam cracking, a gaseous or liquid hydrocarbon feed is diluted with steam and briefly heated to a high temperature in a furnace without the presence of oxygen. Steam crackers convert hydrocarbon feedstock to streams rich in light alkenes such as ethylene and propylene and are used as a principal industrial means to generate these valuable petrochemical products. The feed gas in FIG. 1 is composed of separator off gases from various hydroprocessing units within the complex (“flash drum gases”), as well as off gases from the crude unit and stripper column off gases from the hydroprocessing units (“stripper gases”), and it consists of a mix of hydrogen and hydrocarbons (primarily saturated C₁-C₄). The stripper gases are processed in a gas plant that includes a number of columns and other separation apparatus to separate the gases by their number of carbon atoms. Unstabilized gasoline (“wild naphtha”) and lean oil from elsewhere in the complex are also commonly directed to the gas plant to participate in the separation. In a typical configuration, the plant produces a light gas stream that comprises primarily hydrogen and C₂—hydrocarbons (“lean gas”), an LPG (C₃/C₄ hydrocarbons) stream (that may be further separated into individual C₃ and C₄ hydrocarbon streams), and a naphtha stream that comprises primarily heavier hydrocarbons. The lean oil also absorbs hydrocarbons and thus becomes enriched (“rich oil”). The lean gas from the plant is afterward sent to the steam cracker. The other streams from the plant may be eventually fed to the steam cracker as well. The flash drum gases are at fairly high pressure (791 to 3204 kPa, 100 to 450 psig) and are relatively concentrated in hydrogen (30-95 mol %). These are blended and sent to a pressure-swing adsorption (PSA) unit to produce high-purity (99.9 mol %) hydrogen and a tail gas stream that is used for fuel gas in many typical designs. However, this can waste a lot of potential value, as the fuel gas contains an appreciable amount of C₂+ hydrocarbons that would be quite valuable as steam cracker feed.

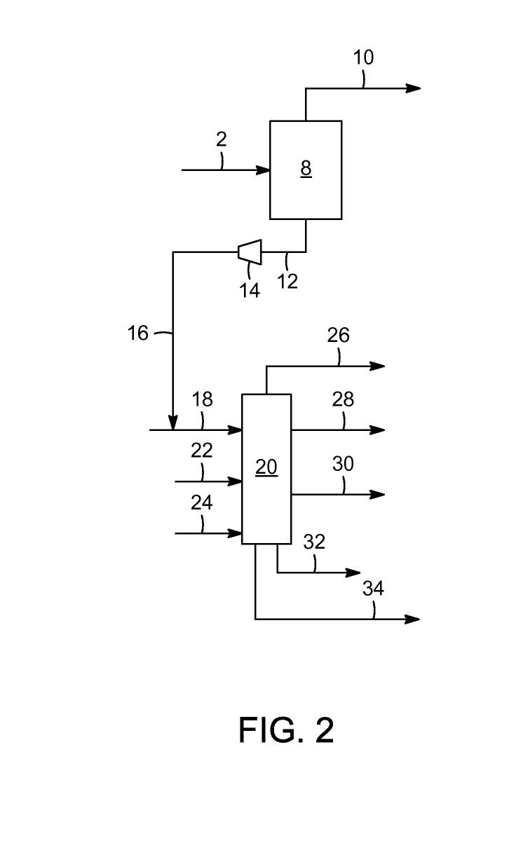

This invention proposes a new flow scheme (FIG. 2), in which the PSA tail gas is not used for fuel gas but is instead compressed using the existing tail gas compressor and mixed with the stripper gases for processing in the gas plant. This modification enables the recovery of a significant portion of C₂+ hydrocarbon material within the tail gas as valuable steam cracker feed. A considerable net product value gain can be obtained with the new case (FIG. 2) versus the base scheme (FIG. 1). The enhanced recovery is achieved at a penalty to the gas plant and steam cracker costs because of the additional gas processing requirements but the added costs are estimated to be minor when compared to the advantages achieved by sending C₂+ hydrocarbons to the stream cracker instead of being burned as fuel gas.

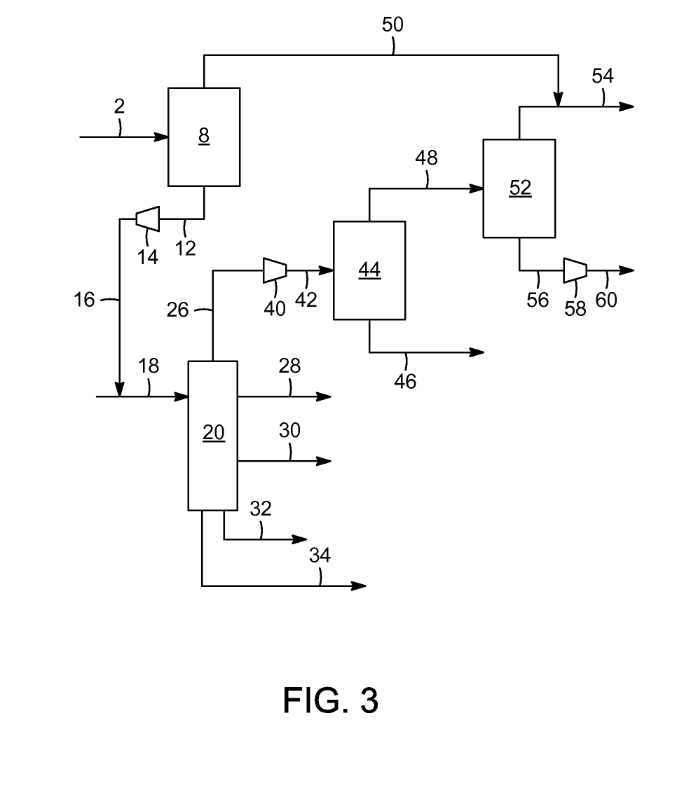

A significant change to the base scheme (FIG. 1) is proposed by the present invention. An additional embodiment of the invention as shown in FIG. 3 further processes the lean gas from the gas plant. Instead of sending the lean gas to the steam cracker as in FIG. 2, the lean gas is compressed and sent through a multi-stage PSA scheme to produce a high purity hydrogen stream, a tail gas stream comprising mostly hydrogen/methane that can be used as fuel gas, and a tail gas stream comprising mostly C₂+ hydrocarbons that can be used as steam cracker feed. This additional processing could result in greater hydrogen recovery and also significantly unload hydrogen/methane from the steam cracker, which would be beneficial since hydrogen/methane waste capacity and energy without being converted into high-value petrochemical products in the steam cracker.

This invention provides a means to enhance the feed to a steam cracker within an integrated refinery and steam cracker complex at the expense of reduction of fuel gas production. Pressure swing adsorption (PSA) provides an efficient and economical means for separating a multi-component gas stream containing at least two gases having different adsorption characteristics. The more strongly adsorbable gas can be an impurity which is removed from the less strongly adsorbable gas which is taken off as product; or, the more strongly adsorbable gas can be the desired product, which is separated from the less strongly adsorbable gas. In PSA, a multi-component gas is typically fed to at least one of a plurality of adsorption zones at an elevated pressure effective to adsorb at least one component, while at least one other component passes through. At a defined time, the feed stream to the adsorber is terminated and the adsorption zone is depressurized by one or more co-current depressurization steps wherein pressure is reduced to a defined level which permits the separated, less strongly adsorbed component or components remaining in the adsorption zone to be drawn off without significant concentration of the more strongly adsorbed components. Then, the adsorption zone is depressurized by a counter-current depressurization step wherein the pressure on the adsorption zone is further reduced by withdrawing desorbed gas counter-currently to the direction of the feed stream. Finally, the adsorption zone is purged and repressurized. The combined gas stream produced during the counter-current depressurization step and the purge step is typically referred to as the tail gas stream. The final stage of repressurization is typically performed by introducing a slipstream of product gas comprising the lightest gas component produced during the adsorption step. This final stage of repressurization is often referred to as product repressurization. In multi-zone systems, there are typically additional steps, and those noted above may be done in stages. Various classes of adsorbents are known to be suitable for use in PSA systems, the selection of which is dependent upon the feedstream components and other factors. Molecular sieves such as the microporous crystalline zeolite and non-zeolitic catalysts, particularly aluminophosphates (AlPO) and silicoaluminophosphates (SAPO), are known to promote reactions such as the conversion of oxygenates to hydrocarbon mixtures.

FIG. 1 shows a base gas processing flow scheme that has previously been employed. Flash drum gas blend 2 that comprises 81 mol % hydrogen, 2 mol % hydrogen sulfide, less than 1 mol % water, 8 mol % methane and 9 mol % C₂+ hydrocarbons is sent to a pressure swing adsorption unit 8. In this example, there is 139 MT/day and 18.2 MT-mole/day flash drum gas blend from hydroprocessor units in a refinery. From the PSA unit 8, there is a pure hydrogen stream 10 that is 99.9% hydrogen that is produced at a rate of 27 MT/day and 13.3 MT-mole/day. A fuel gas stream 12 is shown exiting through compressor 14 to compressed fuel gas stream 16. The fuel gas stream comprises 30 mol % hydrogen, 8 mol % hydrogen sulfide, 2 mol % water, 28 mol % methane and 32 mol % C₂+ hydrocarbons.

A stripper gas blend 18, a wild naphtha stream 22 and a lean oil stream 24 is shown being sent to gas plant 20. The stripper gas blend comprises 59 mol % hydrogen, 5 mol % hydrogen sulfide, 1 mol % water, 7 mol % methane and 28 mol % C₂+ hydrocarbons that is at a flow rate of 232 MT/day, 12.4 MT-mole/day. The wild naphtha stream 22 is at a rate of 9060 MT/day and 96 MT-mole/day and lean oil stream 24 is at 559 MT/day and 3.6 MT-mole/day. The product streams produced from the gas plant are lean gas stream 80 (124 MT/day, 11.1 MT-mole/day), C₃ hydrocarbons stream 82 (142 MT/day, 3.2 MT-mole/day), C₄ hydrocarbons stream 84 (671 MT/day, 11.5 MT-mole/day), rich oil stream 86 (613 MT/day, 4.6 MT-mole/day) and naphtha stream 88 (8298 MT/day, 82 MT-mole/day). The lean gas, C₃ hydrocarbons, C₄ hydrocarbons, rich oil and naphtha are sent to a steam cracker (not shown).

The lean gas 80 comprises 66 mol % hydrogen, 9 mol % hydrogen sulfide, less than 1 mol % water, 10 mol % methane and 15 mol % C₂+ hydrocarbons.

FIG. 2 provides a flow scheme in which PSA tail gas is sent to a gas plant. A flash drum gas blend 2 that comprises 81 mol % hydrogen, 2 mol % hydrogen sulfide, less than 1 mol % water, 8 mol % methane and 9 mol % C₂+ hydrocarbons is sent to a pressure swing adsorption unit 8. In this example, there is 139 MT/day and 18.2 MT-mole/day flash drum gas blend from hydroprocessor units in a refinery. From the PSA unit 8, there is a pure hydrogen stream 10 that is 99.9 mol % hydrogen that is produced at a rate of 27 MT/day and 13.3 MT-mole/day. A stream 12 is sent to a compressor 14 to stream 16 to be combined with stripper gas blend 18. The stripper gas blend 18 which is at 40° C. (104° F.) and 584 kPa (70 psig) comprises 59 mol % hydrogen, 5 mol % hydrogen sulfide, 1 mol % water, 7 mol % methane and 28 mol % C₂+ hydrocarbons. Stream 18 is sent to gas plant 20 where a series of fractionation columns and other separation apparatus separates a stream into a number of hydrocarbon streams including lean gas stream 26 (190 MT/day, 15.2 MT-mole/day) that comprises 58 mol % hydrogen, 9 mol % hydrogen sulfide, less than 1 mol % water, 16 mol % methane and 16 mol % C₂+ hydrocarbons. Also shown being sent to gas plant 20 is wild naphtha stream 22 (9060 MT/day, 96 MT-mole/day) and lean oil stream 24 (766 MT/day, 5.0 MT-mole/day). Also shown are C₃ hydrocarbons stream 28 (153 MT/day, 3.5 MT-mole/day), C₄ hydrocarbons stream 30 (681 MT/day, 11.7 MT-mole/day), rich oil stream 32 (845 MT/day, 6.4 MT-mole/day), and naphtha stream 34 (8297 MT/day, 82 MT-mole/day). Lean gas stream 26, C₃ hydrocarbons stream 28, C₄ hydrocarbons stream 30, rich oil stream 32 and naphtha stream 34 are each sent to a steam cracker (not shown).

in FIG. 3 is shown an alternative flow scheme with lean gas from a gas plant sent to a two-stage PSA train. A flash drum gas blend 2 that comprises 81 mol % hydrogen, 2 mol % hydrogen sulfide, less than 1 mol % water, 8 mol % methane and 9 mol % C₂+ hydrocarbons is sent to a pressure swing adsorption unit 8. From the PSA unit 8, a high quality 99.9 mol % hydrogen stream 50 is produced. A stream 12 is sent to a compressor 14 to stream 16 to be combined with stripper gas blend 18. Stream 18 is sent to gas plant 20 where a series of fractionation columns and other separation apparatus separates a stream into a number of hydrocarbon streams including lean gas stream 26 that in this flow scheme is sent to compressor 40 to compressed gas stream 42 and to a PSA unit 44 that contains an adsorbent to separate hydrogen and methane from C₂+ hydrocarbons. A stream 48 that is mostly hydrogen/methane is sent to another PSA unit 52 which produces a 99.9% pure hydrogen stream 54 that may be combined with hydrogen stream 50 from PSA unit 8. A fuel gas stream 56 that comprises methane is sent to compressor 58 to compressed fuel gas 60.

Also shown is a tail gas 46 that comprises C₂+ hydrocarbons. Also shown are C₃ hydrocarbons stream 28, C₄ hydrocarbons stream 30, rich oil stream 32, and naphtha stream 34. C₃ hydrocarbons stream 28, C₄ hydrocarbons stream 30, rich oil stream 32, and naphtha stream 34 are each sent to a steam cracker (not shown).

The tail gas at low pressure may be sent to the suction of a cracked gas compressor which compresses the gases from the steam cracker furnaces prior to being sent to the product recovery section (pre-treating, cold box and fractionation). The product recovery section will recover the C₂+ paraffin material that is recycled to the steam cracker furnaces. The methane and hydrogen will be separated out by the cold box in the product recovery section. An alternative arrangement is to feed the tail gas product directly to the steam cracker furnaces. This could be done by compressing the tail gas to the pressure required to get it into the furnaces or by designing the PSA tail gas with a pressure sufficient to get it into the steam cracker furnaces. The steam cracker furnace products will then go to the cracked gas compressor and be processed as discussed above.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

### SPECIFIC EMBODIMENTS

While the following is described in conjunction with specific embodiments, it will be understood that this description is intended to illustrate and not limit the scope of the preceding description and the appended claims.

A first embodiment of the invention is a process for treating a gas stream comprising hydrogen, methane and C₂+ hydrocarbons comprising (a) sending the gas stream to at least one pressure swing adsorption unit to produce a high quality hydrogen stream and a second gas stream comprising hydrogen, methane and C₂+ hydrocarbons; and (b) sending the second gas stream comprising hydrogen, methane and C₂+ hydrocarbons to a gas plant to be separated into a plurality of product streams. An embodiment of the invention is one, any or all of prior embodiments in this paragraph up through the first embodiment in this paragraph wherein the second gas stream comprising hydrogen, methane and C₂+ hydrocarbons is compressed before going to the gas plant. An embodiment of the invention is one, any or all of prior embodiments in this paragraph up through the first embodiment in this paragraph wherein the plurality of product streams comprise a C₃ hydrocarbons stream, a C₄ hydrocarbons stream, a rich oil stream, a lean gas stream and a naphtha stream. An embodiment of the invention is one, any or all of prior embodiments in this paragraph up through the first embodiment in this paragraph wherein the lean gas stream is sent to a steam cracker. An embodiment of the invention is one, any or all of prior embodiments in this paragraph up through the first embodiment in this paragraph wherein at least one of the lean gas stream, the C₃ hydrocarbons stream, the C₄ hydrocarbons stream, the rich oil stream and the naphtha stream is sent to a steam cracker. An embodiment of the invention is one, any or all of prior embodiments in this paragraph up through the first embodiment in this paragraph wherein the lean gas stream is sent to a pressure swing adsorption unit to produce a hydrogen/methane gas stream and a C₂+ hydrocarbon stream. An embodiment of the invention is one, any or all of prior embodiments in this paragraph up through the first embodiment in this paragraph wherein the hydrogen/methane gas stream is sent to another pressure swing adsorption unit to produce a high quality hydrogen stream and a fuel gas stream. An embodiment of the invention is one, any or all of prior embodiments in this paragraph up through the first embodiment in this paragraph wherein the C₂+ hydrocarbon stream is sent to a steam cracker.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for treating a gas stream comprising hydrogen, methane and C₂+ hydrocarbons comprising:
(a) sending said gas stream to at least one pressure swing adsorption unit to produce a high quality hydrogen stream and a second gas stream comprising hydrogen, methane and C₂+ hydrocarbons; and
(b) sending said second gas stream comprising hydrogen, methane and C₂+ hydrocarbons to a gas plant to be separated into a plurality of product streams.

2. The process of claim 1 wherein said second gas stream comprising hydrogen, methane and C₂+ hydrocarbons is compressed before going to said gas plant.

3. The process of claim 1 wherein said plurality of product streams comprise a C₃ hydrocarbons stream, a C₄ hydrocarbons stream, a rich oil stream, a lean gas stream and a naphtha stream.

4. The process of claim 3 wherein said lean gas stream is sent to a steam cracker.

5. The process of claim 3 wherein at least one of said lean gas stream, said C₃ hydrocarbons stream, said C₄ hydrocarbons stream, said rich oil stream and said naphtha stream is sent to a steam cracker.

6. The process of claim 3 wherein said lean gas stream is sent to a pressure swing adsorption unit to produce a hydrogen/methane gas stream and a C₂+ hydrocarbon stream.

7. The process of claim 6 wherein said hydrogen/methane gas stream is sent to another pressure swing adsorption unit to produce a high quality hydrogen stream and a fuel gas stream.

8. The process of claim 6 wherein said C₂+ hydrocarbon stream is sent to a steam cracker.
